(19) United States
(12) Patent Application Publication
(10) Pub. No.: US 2011/0054200 A1
(43) Pub. Date: Mar. 3, 2011

(54) Systems and Processes for Biodiesel Production

(75) Inventors: Yang Cai, Ames, IA (US); Jennifer A. Nieweg, Ames, IA (US); Wayne Anthony Turner, Baton Rouge, LA (US); Carla Kern Wilkinson, Ames, IA (US)

(73) Assignee: Catilin, Inc.

(21) Appl. No.: 12/873,211

(22) Filed: Aug. 31, 2010

Related U.S. Application Data
(60) Provisional application No. 61/238,983, filed on Sep. 1, 2009.

(51) Int. Cl. C07C 67/02; B01J 23/90; B01J 8/00

(52) U.S. Cl. 554/169; 422/223; 422/609

(57) ABSTRACT

Described herein are systems and processes for the transesterification of a variety of feedstocks, such as in the form of glyceride-containing vegetable oils, glyceride-containing animal oils, and glyceride-containing algal oils, into biodiesel products using solid, heterogeneous catalysts.

### CROSS REFERENCE TO RELATED APPLICATION

This application claims the benefit of U.S. Provisional Application No. 61/238,983, filed on Sep. 1, 2009, the disclosure of which is incorporated herein by reference in its entirety.

### FIELD OF THE INVENTION

The invention relates generally to biodiesel production. More particularly, the invention relates to biodiesel production using solid, heterogeneous catalysts.

### BACKGROUND

Biodiesel is becoming increasingly useful as a biodegradable, nontoxic alternative to petroleum-based fuels. Examples of biodiesel include soy diesel (or methyl soyate), rapeseed methyl ester, and a variety of other vegetable and animal oil methyl esters. Although interest in biodiesel is increasing, the process by which it is produced has not substantially changed over the years. Biodiesel production typically involves a reaction called “transesterification,” such that an ester is reacted with an alcohol, such as methanol, in the presence of a catalyst to produce a different ester and a different alcohol. Current biodiesel production typically does not allow a catalyst to be recycled, due to its high solubility in methanol. Additionally, the labor and materials involved for neutralization and removal of the catalyst create economic and environmental concerns.

For example, soy diesel is typically prepared commercially by an energy and labor intensive process, in which soybean oil is reacted with methanol in the presence of a homogeneous catalyst, which can be highly toxic. Due to the high solubility of the homogeneous catalyst in methanol, this catalyst cannot be readily recovered or recycled. Also, separation of a desired methyl soyate from the homogeneous catalyst and other co-products typically involves precise neutralization with strong acids, such as hydrochloric acid (or HCl), and extensive washes with water to remove a resulting salt. Glycerol (or glycerin) is a valuable co-product of transesterification, and has a variety of cosmetic, industrial, and food uses. However, the wet washing operation used to separate the methyl soyate tends to introduce impurities into glycerol, which can complicate its separation. In particular, separation of glycerol from the resulting salt is typically carried out by vacuum distillation. Because glycerol has a relatively high boiling point, vacuum distillation becomes a costly and energy intensive operation.

It is against this background that a need arose to develop the systems and processes for biodiesel production described herein.

### SUMMARY

Certain aspects of the invention relate to processes for producing biodiesel. In one embodiment, a process includes: (1) preparing a catalyst mixture that includes a solid, heterogeneous catalyst and a (C₁-C₅)alcohol; (2) combining the catalyst mixture with a glyceride-containing feedstock to provide a reaction mixture; (3) reacting the reaction mixture to produce glycerol and a fatty acid (C₁-C₅)alkyl ester; (4) recovering the catalyst from the reaction mixture; (5) recovering an unreacted portion of the (C₁-C₅)alcohol from the reaction mixture; (6) separating the glycerol from the reaction mixture; and (7) separating the fatty acid (C₁-C₅)alkyl ester from the reaction mixture.

Additional aspects of the invention relate to devices and systems for carrying out such a process. In one embodiment, a biodiesel production system includes: (1) at least one reactor configured to react a reaction mixture to produce glycerol and a fatty acid (C₁-C₅)alkyl ester, wherein the reaction mixture includes a solid, heterogeneous catalyst, a (C₁-C₅)alcohol, and a glyceride-containing feedstock; (2) at least one catalyst recovery unit coupled to the reactor and configured to recover the catalyst from the reaction mixture and to recycle the catalyst back to the reactor; (3) at least one glycerol separator coupled to the reactor and configured to separate a first phase including the glycerol from a second phase including the fatty acid (C₁-C₅)alkyl ester; (4) a first alcohol stripper coupled to the glycerol separator and configured to recover a first unreacted portion of the (C₁-C₅)alcohol from the first phase; (5) a second alcohol stripper coupled to the glycerol separator and configured to recover a second unreacted portion of the (C₁-C₅)alcohol from the second phase; and (6) a biodiesel purification unit coupled to the second alcohol stripper and configured to separate the fatty acid (C₁-C₅)alkyl ester from the second phase.

Other aspects and embodiments of the invention are also contemplated. The foregoing summary and the following detailed description are not meant to restrict the invention to any particular embodiment but are merely meant to describe some embodiments of the invention.

### BRIEF DESCRIPTION OF THE DRAWINGS

For a better understanding of the nature and objects of some embodiments of the invention, reference should be made to the following detailed description taken in conjunction with the accompanying drawings, in which:

FIG. 1 illustrates a system for producing biodiesel, according to an embodiment of the invention; and

FIG. 2 illustrates a system for producing biodiesel, according to another embodiment of the invention.

### DETAILED DESCRIPTION

### Definitions

The following definitions apply to some of the aspects described with respect to some embodiments of the invention. These definitions may likewise be expanded upon herein.

As used herein, the singular terms “a,” “an,” and “the” include plural referents unless the context clearly dictates otherwise. Thus, for example, reference to an object can include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the terms “optional” and “optionally” mean that the subsequently described event or circumstance may or may not occur and that the description includes instances where the event or circumstance occurs and instances in which it does not.

As used herein, the term “size” refers to a characteristic dimension of an object. Thus, for example, a size of an object that is spherical can refer to a diameter of the object. In the case of an object that is non-spherical, a size of the object can refer to an average of various orthogonal dimensions of the object. Thus, for example, a size of an object that is a spheroidal can refer to an average of a major axis and a minor axis of the object. When referring to a set of objects as having a specific size, it is contemplated that the objects can have a distribution of sizes around the specific size. Thus, as used herein, a size of a set of objects can refer to a typical size of a distribution of sizes, such as an average size, a median size, or a peak size.

As used herein, the terms “couple,” “coupled,” and “coupling” refer to an operational connection or linking. Coupled objects can be directly connected to one another or can be indirectly connected to one another, such as through another set of objects.

### Heterogeneous Catalysts for Biodiesel Production

Certain embodiments of the invention relate to the use of solid, heterogeneous catalysts for biodiesel production. A variety of solid, heterogeneous catalysts can be used, such as a variety of calcium-containing catalysts. Examples of calcium-containing catalysts include catalysts in a particulate or powder form and that include calcium or calcium-containing moieties, such as calcium oxide (or CaO) or calcium carbonate (or CaCO₃). Calcium or calcium-containing moieties can be incorporated within a suitable matrix or can be used without such matrix. Additional examples of calcium-containing catalysts include kiln dusts and other calcium-containing dusts. Certain aspects of kiln dusts are described in the U.S. patent application of Lin et al., entitled “Solid Catalyst System for Biodiesel Production,” published as US 2009/0112007, the disclosure of which is incorporated herein by reference in its entirety. While certain embodiments are described with reference to solid, heterogeneous catalysts, it is also contemplated that semi-heterogeneous or homogeneous catalysts can be used in place of, or in combination with, solid, heterogeneous catalysts.

Advantageously, a calcium-containing catalyst can be a substantially insoluble, heterogeneous catalyst that is readily recovered from a reaction mixture, without wet washing or neutralization, thereby facilitating separation of biodiesel products and valuable co-products, such as glycerol. In conjunction, the calcium-containing catalyst is readily recycled for use in subsequent catalytic reactions. Also, the calcium-containing catalyst is readily activated, and is stable, even after repeated use in catalytic reactions. Moreover, the calcium-containing catalyst is highly active, producing biodiesel products from a variety of feedstocks rapidly and under moderate conditions similar to those for homogeneous catalysts.

A catalytic activity of a calcium-containing catalyst can at least partially correspond to, or at least partially derive from, calcium or a calcium-containing moiety present in the catalyst. For some embodiments, a calcium-containing catalyst can include from about 10 weight percent (or wt. %) to about 80 wt. % of calcium, such as from about 10 wt. % to about 50 wt. %, from about 15 wt. % to about 65 wt. %, from about 20 wt. % to about 60 wt. %, or from about 30 wt. % to about 40 wt. % of calcium. Calcium can be in the form of calcium oxide (or CaO), calcium carbonate (or CaCO₃), calcium sulfate (or CaSO₄), calcium hydroxide (or Ca(OH)₂), or a combination thereof. For some embodiments, a calcium-containing catalyst can include more CaO than any other single alkaline earth metal moiety, and can include at least about 15 wt. % of CaO, such as at least about 30 wt. %, at least about 45 wt. %, at least about 50 wt. %, or at least about 55 wt. % of CaO, and up to about 95 wt. ° A) of CaO. In addition to calcium-containing moieties, a calcium-containing catalyst can include sodium, potassium, magnesium, quartz (or SiO₂), or a combination thereof.

A calcium-containing catalyst is desirably fine-grained, with a large surface area to enhance contact with a feedstock during catalytic reaction. A surface area of a calcium-containing catalyst can vary, depending upon the type of calcium-containing catalyst selected for biodiesel production. For some embodiments, a surface area of a calcium-containing catalyst can be in the range of about 0.05 m²/g to about 10 m²/g, such as from about 0.05 m²/g to about 5 m²/g, from about 0.1 m²/g to about 5 m²/g, from about 0.3 m²/g to about 3 m²/g, or from about 0.1 m²/g to about 2 m²/g. In addition, a calcium-containing catalyst can be recovered and reused multiple times, such as 5, 10, 15, 17, 20, or more times. Even after its catalytic activity declines, such as after about 15 to 20 reaction cycles, a calcium-containing catalyst can be substantially regenerated to full catalytic activity by calcination at a suitable temperature.

For example, a calcium-containing dust can serve as a highly active, heterogeneous catalyst for transesterification of a variety of feedstocks to produce biodiesel and glycerol. A calcium-containing dust is typically alkaline, and is typically fine-grained, including particles having sizes in the range of about 0.1 micrometer (or μm) to about 100 μm and a specific gravity in the range of about 2.6 to about 2.8. The particles can be partially calcinated and untreated raw feed, clinker dust, and fuel ash, enriched with sulfates, halides, and other volatiles. One type of calcium-containing dust typically has about 38 wt. % of calcium, where at least about 80 percent of the calcium is in the form of CaO. Another type typically has about 31 wt. % of calcium, where at least about 80 percent of the calcium is in the form of CaCO₃. A further type typically has about 40 wt. % of calcium, where at least about 50 percent of the calcium is in the form of CaO, and the remaining calcium is substantially in the form of calcite, calcium silicates, or a combination thereof.

A calcium-containing dust can vary chemically, depending upon whether high-calcium lime, such as chemical lime, hydrated lime, or quicklime; or dolomitic lime is manufactured. A resulting calcium-containing dust is typically alkaline, and is typically fine-grained, including particles having sizes in the range of about 50 nanometer (or nm) to about 2 centimeter (or cm), such as from about 100 nm to about 3 millimeter (or mm).

Other types of solid, heterogeneous catalysts can be used, such as a porous silica-metal oxide composite catalyst that can provide catalytic activity and other benefits similar to those of a calcium-containing dust. Certain aspects of composite catalysts are described in the PCT patent application of Lin et al., entitled “New Composite-Based Catalysts for Biodiesel Production,” published as WO 2008/013551, the disclosure of which is incorporated herein by reference in its entirety. Composite catalysts can be used in combination with calcium-containing dusts to yield catalysts having further enhanced activity.

A catalytic activity of a composite catalyst can at least partially correspond to, or at least partially derive from, an alkaline earth metal or an alkaline earth metal-containing moiety present in the composite catalyst. An alkaline earth metal can be present along with silicon in a composite matrix of silicon and oxygen atoms, in which a fraction of silicon atoms are replaced by alkaline earth metal atoms. For some embodiments, the alkaline earth metal is calcium, and, for other embodiments, the alkaline earth metal is barium or magnesium. A composite catalyst can be produced from a variety of ratios of silicon-containing reagents, such as orthosilicates, and alkaline earth metal-containing reagents, such as CaO, BaO, MgO, Ca(OH)₂, and Mg(OH)₂. A resulting composite catalyst can have a molar ratio of silicon to alkaline earth metal in the range of about 1/10 to about 10/1, such as from about 1/1 to about 10/1 or from about 1/1 to about 6/1.

A composite catalyst is desirably in the form of porous particles having sizes in the range of about 1 nm to about 50 nm, such as from about 1 nm to about 20 nm, and with large surface areas due to their porosity. For some embodiments, a composite catalyst includes both acidic and alkaline sites, and a surface area of the composite catalyst can be greater than about 50 m²/g, such as greater than about 200 m²/g, greater than about 400 m²/g, or greater than about 800 m²/g. In particular, the composite catalyst can have a surface area in the range of about 200 m²/g to about 1,000 m²/g, such as from about 250 m²/g to about 900 m²/g, from about 250 m²/g to about 300 m²/g, from about 400 m²/g to about 500 m²/g, or from about 800 m²/g to about 950 m²/g. Pores of a composite catalyst can have sizes in the range of about 1 nm to about 20 nm, such as from about 1 nm to about 10 nm, from about 1 nm to about 2 nm, from about 2 nm to about 3 nm, or from about 8 nm to about 10 nm.

### Biodiesel Production

Described as follows are systems and processes for the transesterification of a variety of feedstocks, such as in the form of glyceride-containing vegetable oils and glyceride-containing animal oils, into biodiesel products, such as in the form of fatty acid alkyl esters. Advantageously, the systems and processes address a number of technical challenges and provide a number of benefits. In particular, by using a solid, heterogeneous catalyst, the catalyst can be readily recovered from a reaction mixture, without wet washing or neutralization, thereby facilitating separation of biodiesel products and other valuable co-products with a high degree of purity, such as glycerol. The recovered catalyst can be readily recycled for use in subsequent catalytic reactions, thereby allowing the transesterification to be carried out with reduced costs and reduced environmental concerns. Also, the transesterification can occur rapidly and under moderate conditions of temperature and pressure, with a feedstock conversion rate reaching at least about 98 percent, at least about 98.5 percent, or at least about 99 percent within 2 or 3 hours. Moreover, the transesterification can be carried out in a substantially continuous manner for improved efficiency and throughput, thereby rendering it suitable for implementation in commercially viable plants. However, it is also contemplated that the transesterification can be carried out in a batch manner or a semi-continuous manner.

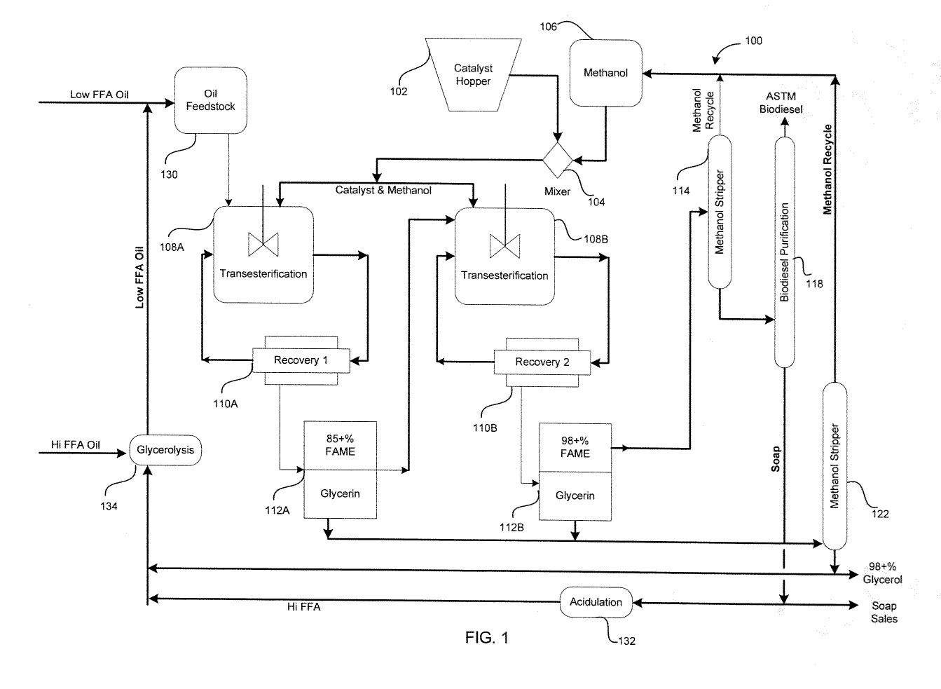

One of a variety of systems for biodiesel production is illustrated in FIG. 1 and is described in the following in accordance with an embodiment of the invention. In particular, FIG. 1 illustrates a biodiesel production system 100, which is implemented for operation in a substantially continuous manner.

Referring to the biodiesel production system 100 of FIG. 1, a solid, heterogeneous catalyst is introduced into a catalyst hopper 102, or another catalyst storage vessel, and an alcohol is introduced into an alcohol storage vessel 106. The catalyst can include a calcium-containing catalyst, a porous silica-metal oxide composite catalyst, a combination thereof, or any other solid, heterogeneous catalyst. The alcohol can include a (C₁-C₅)alcohol, which includes from 1 to 5 carbon atoms per molecule, or a combination of (C₁-C₅)alcohols. Examples include (C₁-C₂)alcohols including from 1 to 2 carbon atoms per molecule, such as methanol and ethanol; straight-chained or branched (C₃-C₅)alcohols including from 3 to 5 carbon atoms per molecule, such as propanol, iso-propanol, butanol, iso-butanol, sec-butanol, Cert-butanol, pentanol, and sec-pentanol; and combinations thereof. In the illustrated embodiment, the alcohol includes methanol, and a resulting biodiesel product includes a set of fatty acid methyl esters (or FAME's).

A catalyst mixture is prepared by combining the catalyst and methanol, or another (C₁-C₅)alcohol, in a mixer 104. In particular, the reactivity of the catalyst can be enhanced by contacting it with methanol for a sufficient duration of time, prior to catalytic reactions with a feedstock. A duration of alcoholic activation can be relatively short and can be no greater than about 1 hour, such as from about 1 minute to about 1 hour, from about 5 minutes to about 50 minutes, from about 10 minutes to about 45 minutes, from about 15 minutes to about 45 minutes, or for about 30 minutes, or the duration of alcoholic activation can be extended for several hours, such as from about 1 hour to about 3 hours. Further extending the duration of alcoholic activation can sometimes be undesirable, given the potential of leaching of catalytic components into methanol that can reduce the reactivity of the catalyst. Alcoholic activation can be carried out at a temperature at or above room temperature, such as from about 25° C. to about 80° C., from about 35° C. to about 70° C., from about 50° C. to about 65° C., or up to a reflux temperature of methanol, or another (C₁-C₅)alcohol.

The catalyst and methanol are respectively conveyed from the catalyst hopper 102 and the alcohol storage vessel 106 to the mixer 104, by application of pressure, gravity, vacuum, pumps, screw conveyors, belts, magnetic devices, vibrating devices, a combination thereof, or any other mechanism for conveyance. The conveyance of the catalyst and methanol can be controlled to provide effective amounts for catalytic reactions with a feedstock. This can be achieved using any suitable controller, which can be mechanical, electrical, pneumatic, hydraulic, electronic, or a combination thereof. An effective amount of the catalyst can be in the range of about 0.1 wt. % to about 50 wt. %, with respect to a weight of the feedstock, such as from about 0.5 wt. % to about 30 wt. %, from about 1 wt. % to about 20 wt. %, from about 1 wt. % to about 10 wt. %, or from about 1 wt. % to about 6 wt. %, with respect to the weight of the feedstock.

The mixer 104 can be implemented in a variety of ways, and can include a housing or a chamber with a set of inlet ports, a set of outlet ports, and a mechanism to achieve agitation or stirring to prepare the catalyst mixture. Agitation in the mixer 104 can be achieved by a variety of mechanisms, such as mechanical, electrical, pneumatic, hydraulic, sonic, or a combination thereof. The catalyst mixture can be heated and maintained at a desired temperature within the mixer 104 using a heating mechanism, along with a set of sensors and a suitable controller.

As illustrated in FIG. 1, the catalyst mixture is conveyed from the mixer 104 to a set of reactors 108A and 108B, such as by dosing into either, or both, of the reactors 108A and 108B or by any other mechanism for conveyance. Excess methanol, or another (C₁-C₅)alcohol, optionally can be introduced into at least one of the reactors 108A and 108B so as to further drive catalytic reactions towards completion. The excess methanol can be introduced by conveying it from the alcohol storage vessel 106, separately or along with a feedstock into the reactor 108A.

Referring to FIG. 1, the feedstock is conveyed from a feedstock storage vessel 130 to the reactor 108A, such as by dosing into the reactor 108A or by any other mechanism for conveyance. The feedstock can include a glyceride-containing oil, such as a glyceride-containing vegetable oil, a glyceride-containing animal oil, a glyceride-containing algal oil, or a combination thereof, where glycerol is produced as a co-product of transesterification. Examples of glyceride-containing vegetable oils include canola oil, coconut oil, corn oil, cottonseed oil, palm oil, peanut oil, rapeseed oil, soybean oil, and sunflower oil, and examples of glyceride-containing animal oils include fish oil, lard, and tallow. The feedstock can also include a relatively small amount of free fatty acids (or FFA's) corresponding to, or derived from, animal fats, such as poultry fat, typically in an amount up to about 1 wt. % with respect to a weight of the feedstock.

A glyceride present in the feedstock can be in the form of mono-esters of glycerol (or monoglycerides), di-esters of glycerol (or diglycerides), tri-esters of glycerol (or triglycerides), or a combination thereof. A fatty acid moiety of the glyceride can include a C₁₀-C₂₈ alkyl chain, which is a hydrocarbon chain including from 10 to 28 carbon atoms per chain. The C₁₀-C₂₈ alkyl chain can be saturated or can have at least one site of unsaturation, epoxidation, or hydroxylation. For example, the C₁₀-C₂₈ alkyl chain can have from 1 to 4 sites of unsaturation, epoxidation, hydroxylation, or a combination thereof. The C₁₀-C₂₈ alkyl chain can be straight-chained or branched, and can have a variety of intermediate chain lengths, such as C₁₆-C₂₄, C₁₀-C₁₈, C₁₀-C₂₀, C₁₂-C₁₈, C₁₂-C₂₀, C₁₀₋₂₄, or C₁₀₋₂₆. Alkyl chains of other chain lengths are also contemplated, such as C₄-C₃₀ alkyl chains including from 4 to 30 carbon atoms per chain. With respect to the transesterification of a glyceride with a (C₁-C₅)alcohol to produce a fatty acid (C₁-C₅)alkyl ester and glycerol, a glycerol moiety of the glyceride is replaced by a moiety corresponding to, or derived from, the (C₁-C₅)alcohol, thus liberating glycerol from the glyceride. In the case of methanol, the glycerol moiety of the glyceride is replaced by a methyl moiety corresponding to, or derived from, methanol, thus liberating glycerol from the glyceride and producing a set of FAME's.

It is contemplated that the feedstock can be pre-conditioned or pre-treated, prior to its introduction into the reactor 108A, so as to render it suitable for transesterification, such as by removal of moisture and impurities, dissolving, filtering, heating, purging, or a combination thereof. For example, the feedstock can be contacted with a molecular sieve, a desiccant, or another suitable absorbent for water and other undesired gases and liquids. Examples of molecular sieves include those corresponding to, or derived from, aluminosilicate minerals, clays, porous glasses, microporous charcoals, zeolites, active carbons, and synthetic compounds that have open structures or pores. As another example of feedstock pre-treatment, FFA's can be stripped using steam or caustic soda.

In the illustrated embodiment, the reactors 108A and 108B are arranged in a multi-stage configuration, with the reactor 108A serving as a first stage reactor, and the reactor 108B serving as a second stage reactor. The use of multiple reactors in a multi-stage configuration can drive transesterification towards completion, by removing glycerol or other co-products after each stage. Also, removal of glycerol can allow faster kinetics, by preventing or reducing instances in which glycerol coats or wraps around the catalyst that can deactivate the catalyst. In addition, the use of multiple reactors can ensure system redundancy so that biodiesel production can be carried out even if a subset of the reactors is inoperative or is being serviced. While two reactors and two stages are illustrated in FIG. 1, it is contemplated that additional reactors and additional stages can be included for other embodiments. For example, other embodiments can include three reactors and three stages, four reactors and four stages, or more.

The reactors 108A and 108B can be implemented in a variety of ways, and each can include a housing or a chamber with a set of inlet ports, a set of outlet ports, and a mechanism to achieve agitation or stirring to ensure substantially uniform blending of components of a reaction mixture and to prevent or reduce settling of the catalyst. The reactors 108A and 108B can be similarly implemented or can be implemented in accordance with different designs. Agitation in the reactors 108A and 108B can be achieved by a variety of mechanisms, such as mechanical, electrical, pneumatic, hydraulic, sonic, or a combination thereof. For example, either, or both, of the reactors 108A and 108B can be implemented as a continuously stirred tank or a sonic mixer. As another example, either, or both, of the reactors 108A and 108B can be implemented as a pressurized reactor with a set of spray nozzles, a set of impellers, and a set of internal compartments to ensure intimate blending of the feedstock, the catalyst, and methanol. Each of the reactors 108A and 108B can also include a mechanism to achieve recirculation of a reaction mixture in a controlled and substantially continuous manner.

Advantageously, transesterification can be carried out within the reactors 108A and 108B under moderate conditions of temperature and pressure, such as at a temperature from about 40° C. to about 100° C., from about 50° C. to about 80° C., or from about 60° C. to about 70° C., and at or near atmospheric pressure, such as from about 0.5 atmosphere to about 2 atmosphere, from about 0.5 atmosphere to about 1.5 atmosphere, or from about 0.8 atmosphere to about 1.2 atmosphere. Depending on reaction conditions, transesterification can be carried out at other temperatures and pressures, such as at a pressure greater than about 2 atmosphere. A reaction mixture can be heated and maintained at a desired temperature and pressure within the reactors 108A and 108B using a heating and pressure control mechanism, along with a set of sensors and a suitable controller.

Referring to FIG. 1, each of the reactors 108A and 108B is arranged along with a catalyst recovery unit 110A or 110B and a glycerol separator 112A or 112B in the multi-stage configuration. In particular, transesterification proceeds in the reactor 108A, and a reaction mixture forward flow is removed from a recirculation stream and is subjected to a filtration mechanism included within the catalyst recovery unit 110A. The filtration mechanism allows recovery of the catalyst present in the reaction mixture forward flow, which recovered catalyst is then recycled back into the reactor 108A for further catalytic reactions. The reaction mixture forward flow is next conveyed to the glycerol separator 112A, which substantially removes or separates glycerol from the reaction mixture forward flow. The separated glycerol, along with any unreacted methanol and any other unreacted or catalytic components, are conveyed to an alcohol stripper 122, which is further described below, and the separated glycerol can be recycled back for further catalytic reactions or can be recovered for other uses. After separation of glycerol by the glycerol separator 112A, a remaining portion of the reaction mixture forward flow can include a relatively high percentage of a biodiesel product, such as from about 70 wt. % to about 90 wt. % of a set of FAME's, from about 75 wt. % to about 85 wt. % of the set of FAME's, from about 80 wt. % to about 90 wt. % of the set of FAME's, or at least about 85 wt. % of the set of FAME's.

The reaction mixture forward flow is next conveyed to the reactor 108B to substantially complete the transesterification, along with operations by the catalyst recovery unit 110B and the glycerol separator 112B similar to those described for the catalyst recovery unit 110A and the glycerol separator 112A. After separation of glycerol by the glycerol separator 112B, a resulting reaction mixture forward flow can include an even higher percentage of a biodiesel product, such as from about 90 wt. % to about 100 wt. % of a set of FAME's, from about 95 wt. % to about 99.9 wt. % of the set of FAME's, or at least about 98 wt. % or at least about 99 wt. % of the set of FAME's.

Reaction conditions in the reactor 108E can be adjusted or modified, relative to those in the reactor 108A, to account for changes in chemical composition as the transesterification proceeds towards completion. In particular, a concentration of methanol, or another (C₁-C₅)alcohol, can be controlled so as to be at a relatively higher level in the reactor 108A and a relatively lower level in the reactor 108B. For some embodiments, the concentration of methanol in the reactor 108A can be maintained in the range of about 8 volume percent (or vol. %) to about 20 vol. %, such as from about 8 vol. % to about 17 vol. % or from about 10 vol. % to about 15 vol. %, while the concentration of methanol in the reactor 108B can be maintained in the range of about 2 vol. % to about 10 vol. %, such as from about 2 vol. % to about 8 vol. % or from about 4 vol. % to about 6 vol. %. These ranges of methanol concentration allow the transesterification to be driven towards completion, while avoiding or reducing excessive amounts of methanol in the reactor 108E that can carry glycerol and complicate separation of a biodiesel product.

The catalyst recovery units 110A and 110B can be implemented in a variety of ways, and can be similarly implemented or can be implemented in accordance with different designs. Filtration by the catalyst recovery units 110A and 110E can be achieved by a variety of mechanisms, such as a set of sintered metal tubes, a set of candle filters, a set of centrifuges, or a combination thereof. The glycerol separators 112A and 112B can be implemented in a variety of ways, and can be similarly implemented or can be implemented in accordance with different designs. Separation of glycerol by the glycerol separators 112A and 112B can be achieved by a variety of mechanisms, such as a set of decanters, a set of vertical or horizontal settling vessels, a set of coalescers, a set of centrifuges, or a combination thereof. While not illustrated in FIG. 1, it is contemplated that, in the case of a substantially continuous process, a recirculation stream from either, or both, of the reactors 108A and 108B can be processed to recover and recycle the catalyst, and a reaction mixture can be conveyed to a plug flow reactor that is externally heated to substantially convert the feedstock to a set of FAME's. The catalyst can be recovered and recycled a number of times.

Referring to FIG. 1, the reaction mixture forward flow is conveyed from the glycerol separator 112B to an alcohol stripper 114, while the separated glycerol from either, or both, of the glycerol separators 112A and 112B is conveyed to the alcohol stripper 122. The alcohol stripper 114 substantially removes or recovers any unreacted methanol, or another (C₁-C₅)alcohol, from the reaction mixture forward flow, such as down to a concentration no greater than about 0.5 vol. %, no greater than about 0.3 vol. %, or below about 0.2 vol. %. The alcohol stripper 114 can be implemented as a vacuum stripper, and recovery of methanol can be achieved by a variety of mechanisms, such as a flash evaporator, a distillation tower, or a combination thereof. For some embodiments, recovery of methanol can be carried out by boiling off methanol at a temperature no greater than about 90° C., such as no greater than about 85° C. or below about 82° C., and at a pressure no greater than about 0.2 atmosphere, such as no greater than about 0.1 atmosphere or no greater than about 0.09 atmosphere. Elevated temperatures, such as greater than about 90° C., can sometimes be undesirable, given the potential of triggering reaction inversion that can reduce a feedstock conversion rate. A desired temperature and pressure can be maintained within the alcohol stripper 114 using a heating and pressure control mechanism, along with a set of sensors and a suitable controller. The recovered methanol is recycled back to the alcohol storage vessel 106 for use in further catalytic reactions. Advantageously, this recycling of methanol can be readily carried out, given the absence of neutralization and wet washing operations that can introduce water and complicate recovery of methanol. In some embodiments, the recovered methanol can be pre-conditioned or pre-treated, prior to its introduction into the alcohol storage vessel 106, so as to render it suitable for catalytic reactions, such as by removal of impurities, dissolving, filtering, heating, purging, or a combination thereof.

After recovery of unreacted methanol by the alcohol stripper 114, a resulting crude biodiesel product is further refined by conveying it through a biodiesel purification unit 118. The biodiesel purification unit 118 substantially removes or separates any remaining co-products, along with any unreacted or catalytic components. The co-products can be in the form of salts of FFA's (or soaps), and removal of the co-products can be achieved by a variety of mechanisms. For example, the crude biodiesel product can be cooled to a temperature below about 21° C., and the co-products can be separated by settling or centrifugation. As another example, the co-products can be separated by either of, or both, distillation and wiped film evaporation.

The separated co-products, along with any unreacted or catalytic components, can be recycled back for further catalytic reactions, can be sold in crude form for animal and poultry feed supplements, or can be refined for a variety of cosmetic, industrial, pharmaceutical, and food uses. Referring to FIG. 1, at least a portion of the co-products is treated by the addition of a set of acids to convert the co-products to FFA's. The conversion of the co-products to FFA's typically involves acidulation, and is carried out in an acidulation unit 132. The resulting FFA's are then conveyed to a glycerolysis unit 134 for conversion to usable glycerides, which can undergo transesterification within the reactors 108A and 108B. In particular, crude glycerol produced from the process is recycled and mixed with the FFA's under suitable conditions to produce glycerides via glycerolysis. A feedstock including a relatively large amount of FFA's, such as greater than about 1 wt. % with respect to a weight of the feedstock, can also be introduced into the glycerolysis unit 134 to convert the FFA's to glycerides. The acidulation unit 132 and the glycerolysis unit 134 can be implemented in a variety of ways, such as including a housing or a chamber with a set of inlet ports, a set of outlet ports, and a mechanism to achieve agitation or stirring.

It is also contemplated that additional operations can be carried out to recover any FFA's or to convert such FFA's to fatty acid alkyl esters. For example, the FFA's can be contacted with a solid acid catalyst so as to catalyze the esterification of the FFA's, and the resulting fatty acid esters can undergo transesterification within the reactors 108A and 108B. Examples of solid acid catalysts include acidic mesoporous aluminum silicate mixed oxides, zeolites, sulfonic-functionalized mesoporous crystalline materials, and sulfonic-functionalized mesoporous silicates.

The biodiesel purification unit 118 substantially removes or separates any remaining impurities, such that a resulting refined biodiesel product satisfies criteria of commercially-acceptable diesel fuel, such as in accordance with specifications of the American Society for Testing and Materials (or ASTM). Removal of impurities can be achieved by a variety of mechanisms. For example, the crude biodiesel product can be subjected to dry washing using a suitable absorbent to remove undesired gases and liquids, followed by filtration to remove undesired solids. As another example, impurities can be separated by either of, or both, distillation and wiped film evaporation. As another example, ion exchange resins can be used to purify crude biodiesel by removing residual salts, soaps, and metals. As a further example, a wet washing operation can also be used.

For some embodiments, the refined biodiesel product can include a residual amount of calcium, which corresponds to, or is derived from, calcium or calcium-containing moieties present in the catalyst used for the transesterification. The residual calcium can be detected by standard techniques, such as Inductively Coupled Plasma (or ICP) Optical Emission Spectroscopy or ICP-Mass Spectroscopy, Atomic Adsorption or Emission Spectroscopy, or Ion Selective Electrode analysis. The refined biodiesel product can include, for example, from about 1 parts per million (or ppm) to about 1,000 ppm of calcium, such as from about 5 ppm to about 500 ppm or from about 50 ppm to about 500 ppm, depending on reaction conditions and the technique used to separate the refined biodiesel product. The residual calcium can be in the form of calcium atoms; calcium ions; calcium-containing compounds, such as CaO or CaCO₃, or a combination thereof. For other embodiments, a residual amount of calcium in the refined biodiesel product can be of such low levels so as to be substantially undetectable.

As illustrated in FIG. 1, the alcohol stripper 122 substantially removes or recovers any unreacted methanol, or another (C₁-C₅)alcohol, from glycerol, such as down to a concentration no greater than about 0.5 vol. %, no greater than about 0.3 vol. %, or below about 0.2 vol. %. The alcohol stripper 122 can be implemented as a vacuum stripper, and recovery of methanol can be achieved by a variety of mechanisms, such as a flash evaporator, a distillation tower, or a combination thereof. For some embodiments, recovery of methanol can be carried out by boiling off methanol at a temperature no greater than about 130° C., such as no greater than about 125° C. or below about 121° C., and at a pressure no greater than about 0.2 atmosphere, such as no greater than about 0.1 atmosphere or no greater than about 0.09 atmosphere. Elevated temperatures, such as greater than about 130° C., can sometimes be undesirable, given the potential of triggering by-product formation that can reduce purity of glycerol. A desired temperature and pressure can be maintained within the alcohol stripper 122 using a heating and pressure control mechanism, along with a set of sensors and a suitable controller. The recovered methanol is recycled back to the alcohol storage vessel 106 for use in further catalytic reactions. Advantageously, this recycling of methanol can be readily carried out, given the absence of neutralization and wet washing operations that can introduce water and complicate recovery of methanol. After recovery of methanol by the alcohol stripper 122, a resulting crude glycerol can be of a relatively high degree of purity, such as from about 90 wt. % to about 100 wt. %, from about 95 wt. % to about 99.9 wt. %, or at least about 97 wt. %. The crude glycerol can be further refined for a variety of cosmetic, industrial, pharmaceutical, and food uses. As previously described, the crude glycerol can also be recycled back to the glycerolysis unit 134 to produce glycerides for further catalytic reactions.

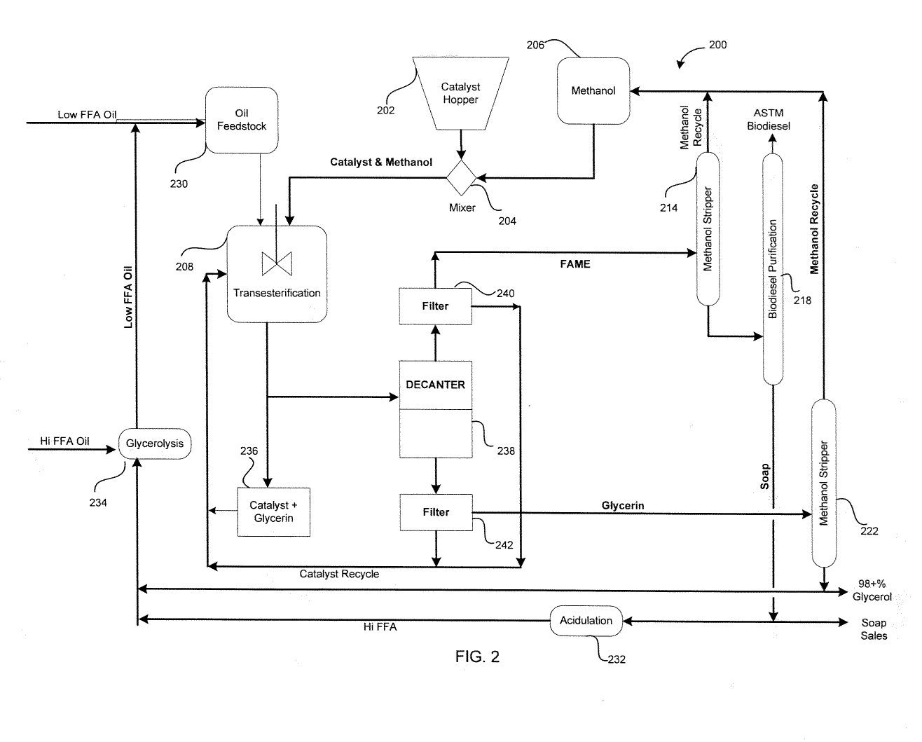

Another of a variety of systems for biodiesel production is illustrated in FIG. 2 and is described in the following in accordance with an embodiment of the invention. In particular, FIG. 2 illustrates a biodiesel production system 200, which is implemented for operation in a batch manner. Certain aspects of the biodiesel production system 200 can be implemented in a similar manner as previously described for the biodiesel production system 100, and those aspects are not repeated below. For example, and like the biodiesel production system 100, the biodiesel production system 200 includes a catalyst hopper 202, a mixer 204, an alcohol storage vessel 206, alcohol strippers 214 and 222, a biodiesel purification unit 218, a feedstock storage vessel 230, an acidulation unit 232, and a glycerolysis unit 234, which can be similarly implemented as their corresponding components of FIG. 1, albeit operated or optimized for batch processing.

Referring to FIG. 2, a catalyst mixture is prepared by combining a catalyst and methanol, or another (C₁-C₅)alcohol, in the mixer 204. For a particular new batch, the catalyst is activated by contacting it with methanol for a sufficient duration of time, prior to catalytic reactions with a feedstock. The catalyst mixture is then conveyed from the mixer 204 to a reactor 208, such as by dosing into the reactor 208 or by any other mechanism for conveyance. Excess methanol, or another (C₁-C₅)alcohol, optionally can be introduced into the reactor 208 so as to further drive catalytic reactions towards completion. The excess methanol can be introduced separately, or along with any catalyst, to maintain an effective amount of the catalyst with respect to a weight of the feedstock, as previously described with reference to FIG. 1. As illustrated in FIG. 2, the feedstock is conveyed from the feedstock storage vessel 230 to the reactor 208, such as by dosing into the reactor 208 or by any other mechanism for conveyance, and a batch production process is initiated.

In the illustrated embodiment, the reactor 208 is arranged in a single-stage configuration, although it is contemplated that multiple reactors and multiple stages can be included for other embodiments. The reactor 208 can be similarly implemented as the reactors 108A and 108B of FIG. 1, and can include a housing or a chamber with a set of inlet ports, a set of outlet ports, and a mechanism to achieve agitation or stirring to ensure substantially uniform blending of components of a reaction mixture and to prevent or reduce settling of the catalyst. To facilitate initiation of transesterification, crude glycerol produced from the process is recycled and introduced into the reactor 208 in an amount in the range of about 0.5 wt. % to about 12 wt. %, with respect to a total weight of the reaction mixture, such as from about 1 wt. % to about 10 wt. %, from about 1 wt. % to about 5 wt. %, or from about 5 wt. % to about 10 wt. %, with respect to the total weight of the reaction mixture. Transesterification is carried out for a duration in the range of about 45 minutes to about 4 hours, such as from about 1 hour to about 3 hours, from about 1 hour to about 2 hours, or from about 2 hours to about 3 hours, until a conversion rate to a set of FAME's is in the range of about 90 percent to about 100 percent, such as from about 95 percent to about 99.9 percent, from about 98.5 percent to about 99.5 percent, or at least about 98.5 percent.

Once the desired conversion rate is reached, agitation in the reactor 208 is halted, and the reaction mixture is allowed to settle for a duration in the range of about 10 minutes to about 2 hours, such as from about 15 minutes to about 1 hour, from about 15 minutes to about 45 minutes, or from about 45 minutes to about 1 hour. Settled components of the reaction mixture are then removed from the reactor 208 and conveyed to a holding vessel 236 to recover a majority of the catalyst, along with a relatively small amount of residual glycerol. The holding vessel 236 can be implemented in a variety of ways, such as a set of vertical or horizontal settling vessels. It is also contemplated that the holding vessel 236 can be more generally implemented as a catalyst recovery unit, and can optionally include a filtration mechanism, such as a set of sintered metal tubes, a set of candle filters, a set of centrifuges, or a combination thereof.

A remaining portion of the reaction mixture is removed from the reactor 208 and conveyed to a decanter 238, which allows separation into FAME and glycerol phases. It is also contemplated that the decanter 238 can be more generally implemented as a glycerol separator, and that separation of the FAME and glycerol phases can be achieved by a variety of other mechanisms, such as a set of vertical or horizontal settling vessels, a set of coalescers, a set of centrifuges, or a combination thereof. As illustrated in FIG. 2, the FAME phase is removed from the decanter 238 and conveyed through a filtration mechanism 240, which allows recovery of any residual catalyst and yields a filtered FAME phase. The filtered FAME phase is then further processed by conveying it through the alcohol stripper 214 and the biodiesel purification unit 218, thereby yielding a refined biodiesel product. The glycerol phase is removed from the decanter 238 and conveyed through a filtration mechanism 242, which allows recovery of any residual catalyst and yields a filtered glycerol phase. The filtered glycerol phase is then conveyed through the alcohol stripper 222, thereby yielding crude glycerol that can be further refined for a variety of uses or can be recycled back to the glycerolysis unit 234 for further catalytic reactions.

Still referring to FIG. 2, the recovered catalyst and glycerol from the holding vessel 236 are recycled back to the reactor 208 for the current batch or for a new batch. Likewise, the recovered catalyst from the filtration mechanisms 240 and 242 is recycled back to the reactor 208 for further catalytic reactions.

While the invention has been described with reference to the specific embodiments thereof, it should be understood by those skilled in the art that various changes may be made and equivalents may be substituted without departing from the true spirit and scope of the invention as defined by the appended claim(s). In addition, many modifications may be made to adapt a particular situation, material, composition of matter, method, or process to the objective, spirit and scope of the invention. All such modifications are intended to be within the scope of the claim(s) appended hereto. In particular, while the methods disclosed herein have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations are not limitations of the invention.

What is claimed is:

1. A process for producing biodiesel, comprising:
preparing a catalyst mixture that includes a solid, heterogeneous catalyst and a (C₁-C₅)alcohol;
combining the catalyst mixture with a glyceride-containing feedstock to provide a reaction mixture;
reacting the reaction mixture to produce glycerol and a fatty acid (C₁-C₅)alkyl ester;
recovering the catalyst from the reaction mixture;
recovering an unreacted portion of the (C₁-C₅)alcohol from the reaction mixture;
separating the glycerol from the reaction mixture; and
separating the fatty acid (C₁-C₅)alkyl ester from the reaction mixture.

2. The process of claim 1, wherein the catalyst includes a calcium-containing catalyst, and the (C₁-C₅)alcohol includes methanol.

3. The process of claim 2, wherein the calcium-containing catalyst includes from 10 wt. % to 80 wt. % of calcium.

4. The process of claim 1, wherein the catalyst includes a porous silica-metal oxide composite catalyst, and the (C₁-C₅)alcohol includes methanol.

5. The process of claim 1, wherein preparing the catalyst mixture includes contacting the catalyst with the (C₁-C₅)alcohol for a duration sufficient to activate the catalyst.

6. The process of claim 5, wherein contacting the catalyst with the (C₁-C₅)alcohol is carried out for a duration in the range of 1 minute to 1 hour and at a temperature in the range of 25° C. to 80° C.

7. The process of claim 1, wherein combining the catalyst mixture with the feedstock is carried out such that an amount of the catalyst is in the range of 0.5 wt. % to 30 wt. %, with respect to a weight of the feedstock.

8. The process of claim 7, wherein the amount of the catalyst is in the range of 1 wt. % to 10 wt. %, with respect to the weight of the feedstock.

9. The process of claim 1, wherein reacting the reaction mixture is carried out using multiple reactors in a multi-stage configuration.

10. The process of claim 1, wherein reacting the reaction mixture is carried out at a temperature in the range of 40° C. to 100° C. and at a pressure in the range of 0.5 atmosphere to 2 atmosphere.

11. The process of claim 1, further comprising recycling the catalyst and the unreacted portion of the (C₁-C₅)alcohol for a subsequent reaction.

12. The process of claim 1, further comprising reacting the glycerol with a free fatty acid to produce an additional, glyceride-containing feedstock.

13. The process of claim 1, wherein separating the fatty acid (C₁-C₅)alkyl ester includes separating a fatty acid salt from the reaction mixture.

14. The process of claim 13, further comprising:
reacting the fatty acid salt with an acid to produce a free fatty acid; and
reacting the glycerol with the free fatty acid to produce an additional, glyceride-containing feedstock.

15. A biodiesel production system, comprising:
at least one reactor configured to react a reaction mixture to produce glycerol and a fatty acid (C₁-C₅)alkyl ester, wherein the reaction mixture includes a solid, heterogeneous catalyst, a (C₁-C₅)alcohol, and a glyceride-containing feedstock;
at least one catalyst recovery unit coupled to the reactor and configured to recover the catalyst from the reaction mixture and to recycle the catalyst back to the reactor;
at least one glycerol separator coupled to the reactor and configured to separate a first phase including the glycerol from a second phase including the fatty acid (C₁-C₅)alkyl ester;
a first alcohol stripper coupled to the glycerol separator and configured to recover a first unreacted portion of the (C₁-C₅)alcohol from the first phase;
a second alcohol stripper coupled to the glycerol separator and configured to recover a second unreacted portion of the (C₁-C₅)alcohol from the second phase; and
a biodiesel purification unit coupled to the second alcohol stripper and configured to separate the fatty acid (C₁-C₅)alkyl ester from the second phase.

16. The biodiesel production system of claim 15, further comprising a mixer coupled to the reactor and configured to combine the catalyst and the (C₁-C₅)alcohol to produce a catalyst mixture and to convey the catalyst mixture to the reactor.

17. The biodiesel production system of claim 16, wherein the first alcohol stripper is coupled to the mixer, such that the first unreacted portion of the (C₁-C₅)alcohol is recycled back to the reactor.

18. The biodiesel production system of claim 17, wherein the second alcohol stripper is coupled to the mixer, such that the second unreacted portion of the (C₁-C₅)alcohol is recycled back to the reactor.

19. The biodiesel production system of claim 15, further comprising a glycerolysis unit coupled to the first alcohol stripper and configured to react the glycerol with a free fatty acid to produce an additional, glyceride-containing feedstock.

20. The biodiesel production system of claim 19, wherein the biodiesel purification unit is configured to separate a soap from the second phase, and further comprising an acidulation unit coupled to the biodiesel purification unit and the glycerolysis unit and configured to react the soap with an acid to produce the free fatty acid.
